(19) United States
(12) Patent Application Publication
(10) Pub. No.: US 2002/0182650 A1
(43) Pub. Date: Dec. 5, 2002

(54) Inhibitors of binding between proteins and macromolecular ligands

(75) Inventors: Michael Sworin, Tyngsboro, MA (US); James C. Jenson, Sudbury, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Polaris Pharmaceuticals, Inc., Sudbury, MA (US)

(21) Appl. No.: 10/082,815

(22) Filed: Feb. 22, 2002

Related U.S. Application Data
(63) Continuation of application No. PCT/US00/23346, filed on Aug. 23, 2000.
(60) Provisional application No. 60/150,230, filed on Aug. 23, 1999.
(60) Provisional application No. 60/150,318, filed on Aug. 23, 1999.
(60) Provisional application No. 60/152,421, filed on Sep. 3, 1999.

(51) Int. Cl. G01N 33/53; G01N 33/542; A61K 31/00

(52) U.S. Cl. 435/7.9; 514/1

(57) ABSTRACT

Disclosed is a compound which inhibits binding between a target protein and a macromolecular ligand of the target protein. The compound comprises a targeting group, an attaching group and, optionally a linker group. In one aspect of the invention, the targeting group is a moiety that binds non-covalently to a surface of the target protein with a Kd of greater than about 0.1 μM and within sufficient proximity to the target protein/macromolecular ligand binding site to inhibit binding between the target protein and the macromolecular ligand. In another aspect of the invention, targeting group is degradable in vivo. In yet another aspect of the invention, the compound comprises a linker group that is cleavable in vivo.

### RELATED APPLICATIONS

This application is a continuation of International Application No. PCT/US00/23346, which designated the United States and was filed on Aug. 23, 2000, published in English, which claims the benefit of U.S. Provisional Application No. 60/150,230, filed Aug. 23, 1999, U.S. Provisional Application No. 60/150,318, filed Aug. 23, 1999 and U.S. Provisional Application No. 60/152,421, filed Sep. 3, 1999. The entire teachings of the International Application and these Provisional Applications are incorporated herein by reference.

The entire teachings of the above application(s) are incorporated herein by reference.

### BACKGROUND OF THE INVENTION

Medicinal chemists have been very successful at developing drugs which modulate the activity of enzymes, which are proteins that catalyze chemical reactions involving one or more substrates. The reaction occurs in a cleft or crevice on the enzyme surface, referred to as an “active site”. The substrate(s) fit into the active site, much as a key fits into a lock. Most known inhibitors are small organic molecules which bind with high affinity to the enzyme by fitting snugly into the active site. As a consequence, substrates are prevented from entering the active site and the enzyme's activity is thereby inhibited. Affinity for the active site can be attained through functional groups on the molecule that are attracted by ionic interactions, Van der Waals forces, hydrogen bonds, hydrophobic interactions and the like to one or more of the functional groups on the active site surface. Because the active site has a three dimensional shape, attractive forces between the molecule and active site can act in multiple directions, further strengthening the binding between the two molecules. Often, enzyme inhibitor binding affinities in the nanomolar to picomolar range can be achieved.

Medicinal chemists have been much less successful in discovering synthetic molecules which can block the binding between proteins and macromolecular ligands at pharmaceutically useful concentrations. The term “macromolecular ligand” refers to a large biological molecule such as a protein, glycoprotein, carbohydrate or nucleic acid which binds to a protein surface at a location other than an enzyme active site. Binding between proteins and macromolecular ligands often causes large biological responses, such as the regulation of gene expression; modulation of cell proliferation, cell secretion, cell migration, immune response, or cell death; chemical modification of proteins such as phosphorylation and dephosphorylation; formation of catalytically active protein complexes; and the like. These processes are critical for the development and maintenance many diseases including cancer and disorders of the immune system, nervous system, circulatory system and others. Treatments for these diseases could be based on drugs which block binding between proteins and their macromolecular ligands. The lack of success in identifying synthetic compounds which block such binding has limited the ability of physicians to effectively treat many diseases.

The development of synthetic molecules which block the binding between the proteins and macromolecular ligands is complicated by the nature of the protein/macromolecule binding site, which is generally large, flat and solvent exposed compared with the cleft or crevice that forms most enzyme active sites. The large flat surface does not provide an inhibitor with the snug fit that is available in a cleft or crevice. In addition, ionic, hydrogen bond, hydrophobic and Van der Waals attractive forces between an inhibitor and a protein/macromolecule binding site can act primarily in only one direction, in contrast with the multidimensional interactions available to an enzyme inhibitor in the enzyme active site. As a result, the vast majority of known synthetic inhibitors of protein/macromolecule binding have weak affinities in the micromolar range, which are far less than the strong, nanomolar to picomolar binding affinity exhibited by most drugs. There is therefore a great need to identify more potent inhibitors of protein/macromolecule binding and for new methodologies for identifying such inhibitors.

### SUMMARY OF THE INVENTION

Applicants have conceived of methodology for identifying potent inhibitors of binding between a target protein and a macromolecular ligand of the target protein. The method is generally applicable to a large number of target proteins, including target proteins which heretofore have no known inhibitors or known inhibitors which are poorly active. Based on this conception, assays for identifying compounds which inhibit protein/macromolecule binding, compounds which inhibit such binding, pharmaceutical compositions comprising such compounds and methods of treating subjects in need of such inhibition and are disclosed herein.

One embodiment of the present invention is a method of identifying a compound which covalently binds to the surface of a target protein in sufficient proximity to the binding site between a macromolecular ligand and the target protein so that the compound inhibits binding of the macromolecular ligand with the target protein. In carrying out the method, a lead compound is selected which non-covalently binds to the surface of a target protein. Preferably, the compound inhibits binding of the macromolecular ligand with the target protein. The lead compound is represented by the structural formula T-H. A plurality of analogs of the lead compound are then prepared, each analog being represented by the structural formula T-L-A. L is a linking group; A is an attaching group; and -L-A, taken together, is different for each analog. In one aspect of the invention, the lead compound binds non-covalently to a surface of the target protein with a Kd of greater than about 0.1 μM. In another aspect of the invention, the lead compound is degradable in vivo. In yet another aspect of the invention, the linker group is cleavable in vivo.

The target protein, the macromolecular ligand and each analog are combined under conditions suitable for binding between the target protein and macromolecular ligand. Each combination is then assayed for inhibition of macromolecular ligand/target protein binding and for covalent binding between the analog and the target protein. Analogs which inhibit macromolecular ligand/target protein binding and which covalently bind with the target protein are then selected for further testing.

Another embodiment of the present invention is a compound which inhibits binding between a target protein and a macromolecular ligand of the target protein. The compound comprises a targeting group, an attaching group and, optionally, a linker group. In one aspect of the invention, the targeting group is a moiety that binds non-covalently to a surface of the target protein with a Kd of greater than about 0.1 μM and within sufficient proximity to the target protein/macromolecular ligand binding site so that the compound inhibits binding between the target protein and the macromolecular ligand. In another aspect of the invention, the targeting group is degradable in vivo. In yet another aspect of the invention, the compound comprises a linker group that is cleavable in vivo.

Another embodiment of the present invention is a method of inhibiting binding between a target protein and a macromolecular ligand in a subject in need of such inhibition. The method comprises the step of administering to the subject an effective amount of a compound described above.

Another embodiment of the present invention is a pharmaceutical composition comprising a compound of the present invention and a pharmaceutically acceptable carrier.

The compounds of the present invention are useful as drugs which can inhibit protein/macromolecular ligand binding or can serve as leads to optimize biological activity or some other pharmacologically relevant property. The compounds disclosed herein are weakly reactive with non-target proteins and therefore are expected to cause minimal side effects. In addition, they are developed from optimizations of compounds known to be weak or modest inhibitors of protein/macromolecular ligand binding.

### BRIEF DESCRIPTION OF THE DRAWINGS

FIG. 1 is a schematic showing a compound of the present invention and its reaction with a target protein. T is a targeting group; X is —H or a blocking group; and A is an attaching group.

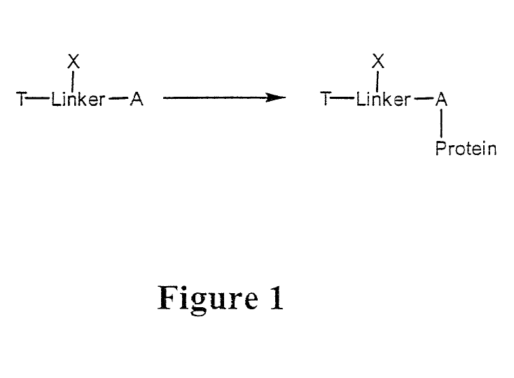

FIG. 2 is a schematic showing a compound of the present invention, said compound comprising a targeting group that is a cleavable or degradable in vivo, and the reaction of the compound with a target protein. T is a targeting group; X is —H or a blocking group; and A is an attaching group.

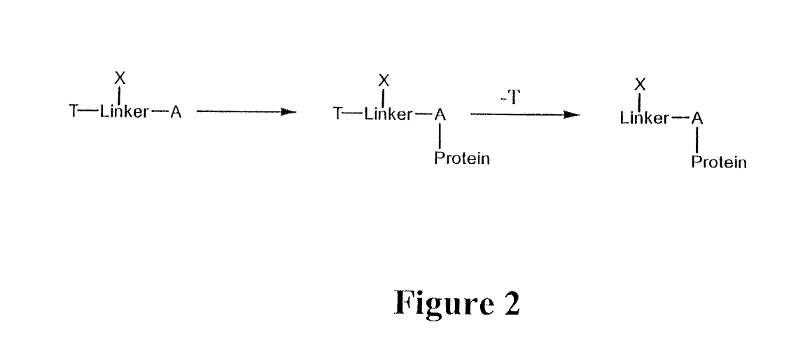

FIG. 3 is a table of amino acid sequences of polypeptide fragments of human monocyte chemoattractant protein-I (SEQ ID NO 10 thru SEQ ID NO 22) which are suitable targeting groups. Cysteinyl residues involved in intramolecular dissulfide bonds are indicated by underlines; the polypeptide having the amino acid sequence of SEQ ID NO. 15 has disulfide bonds between the cysteines at positions 2 and 21 and positions 10 and 16.

### DETAILED DESCRIPTION OF THE INVENTION

The compounds of the present invention comprise a targeting group which binds non-covalently to a surface of a target protein. The targeting group must bind in sufficient proximity to the site at which the target protein binds with its macromolecular ligand so that the binding of the ligand with the target protein can be blocked or inhibited by the compound. Although targeting groups which bind to the target protein with high affinity can be used, high affinity meaning a Dissociation Constant (herein after “Kd”) less than about 0.01 μM, the binding affinity of most targeting groups will be considerably less. As noted above, medicinal chemists have had great difficulty in developing inhibitors of protein/macromolecular binding having a Kd less than 0.1 μM. One advantage of the present invention is that targeting groups can be based on inhibitors which would otherwise be deemed unsuitable for use as drug candidates because of their modest binding affinities. Thus, targeting groups with a Kd for the target protein of greater than 0.1 μM are suitable. In fact, targeting groups with a Kd greater than 1.0 μM will be typical and in many cases greater than 10.0 μM.

The compounds of the present invention also comprise an attaching group in addition to the targeting group. An “attaching group” is a moiety comprising a reactive functional group which can form a covalent bond with an amino acid on the surface of the target protein after the targeting group binds with the target protein. Whereas the targeting group allows the compound to bind non-covalently at or in close proximity to the target protein/macromolecule binding site, the reactive functional group reacts with a moiety on the surface of the target protein, thereby covalently binding the compound to the surface. The covalent bond holds the residue of the compound tightly to the protein surface and provides the affinity required for effective inhibition.

Binding between the target protein and the targeting group “holds” the reactive functional group in a fixed position in space relative to the amino acids on the target protein surface. To form a covalent bond with the surface, the reactive functional group must be in close proximity and in the proper orientation relative to a group on the protein surface with which it can react. Amino acid functional groups which can form a covalent bond with the reactive functional group are referred to herein as “compatible functional groups”; and amino acids comprising a compatible functional group are referred to as “compatible amino acids”. For example, alkyl halides can react with nucleophilic functional groups on lysine, arginine, cysteine or tyrosine, provided that the alkyl halide is held sufficiently close and in a suitable orientation for the displacement of the halide by the nucleophile. Other amino acids comprising compatible functional groups include histidine, tryptophan, serine, threonine, aspartic acid, glutamic acid, methionine, arginine, glutamine, asparagine and the free amino terminus of a protein. The position in space of the reactive functional group can be adjusted, if necessary with a linker group (also referred to as a “linking group”).

Thus, the targeting group and the attaching group can be connected by a linking group. Suitable linking groups do not adversely affect the ability of the targeting group to bind to the protein surface and the ability of the reactive functional group to form a covalent bond with compatible amino acid functional groups on the protein surface. Additionally, the linking group is selected so that after binding between the targeting group and the target protein, the reactive functional group is fixed in three dimensional space at a suitable distance from and in a suitable orientation to a compatible functional group(s) on the protein surface for covalent bond formation.

Because binding between the surface of the target protein and the targeting group “holds” the reactive functional group in close proximity to a compatible amino acid functional group on the protein surface, the reaction rate between the amino acid on the protein surface and the reactive functional group is far greater than the reaction rate between the corresponding free amino acid and the reactive functional group. For example, the drug would react with a compatible amino acid on the target protein surface at a rate of about 10⁻² second⁻¹, assuming a Kd of 10 μM for the targeting group, a reactive functional group with a forward reaction rate of 10⁻⁵ M⁻¹sec⁻¹ with the corresponding free amino acid and a plasma concentration of 100 nM. This rate corresponds to about 1% of the drug per second. Therefore, it is not necessary to have highly reactive attaching groups. In fact, “weakly reactive” attaching groups are preferred because they react minimally with non-target proteins, thereby minimizing the drug's side effects. Thus, the combination of a targeting group with modest binding affinity and a weakly reactive attaching group will provide both selectivity for the target protein and sufficient reactivity to bind with the target protein's surface. A reactive functional group is “weakly reactive”, for example, when the reactive functional group has a forward reaction rate with a free amino acid less than about 10⁻² M⁻¹sec⁻¹ and preferably less than about 10⁻³ M⁻¹sec⁻¹, the free amino acid corresponding to a compatible amino acid. In fact, forward reaction rates less than about 10⁻⁴ M⁻¹sec⁻¹ will be typical and often less than about 10⁻⁵ M⁻¹sec⁻¹. “Forward reaction rate” refers to the rate at which starting materials are converted to product by covalent bond formation between the reactive functional group and a compatible functional group in the side chain of the free amino acid. When the free amino acid contains other functional groups which can form covalent bonds with the reactive functional group, the forward reaction rate is determined after first protecting these other functional groups. The reactive functional group should be reactive enough to form a covalent bond with the compatible amino acid, once the targeting group has bound non-covalently to the protein surface. Typically, the reactive functional group will be sufficiently reactive when the forward reaction rate with the free amino acid is greater than about 10⁻⁸ M⁻¹sec⁻¹ and preferably greater than about 10⁻⁷ M⁻¹sec⁻¹, the free amino acid corresponding to a compatible amino acid.

A “target protein” is a wild type protein which binds with a macromolecular ligand to form a complex which induces one or more biochemical events. A “wild type protein” is a protein that occurs in nature. “Target protein” also includes functionally active fragments of the wild type protein. A “biochemical event” includes the inhibition or initiation of one or more biochemical reactions or a change in the rate at which one or more biochemical reactions occur. Often such binding induces a series of biochemical events. Examples include the up or down regulation of gene expression; stimulation or inhibition of cell proliferation, cell secretion, cell migration, an immune response or cell death; the chemical modification of proteins such as phosphorylation or dephosphorylation; the formation or degradation of catalytically active protein complexes and the like. The phrase “binding between a target protein and macromolecule”, as it is used herein, does not refer to binding between an enzyme and its substrate. Thus, the binding inhibited by the compounds of the present invention refers to binding on the surface of the target protein and not to binding at an enzyme active site. However, an enzyme can have both an active site and a binding site for a macromolecular ligand. Therefore, an enzyme can also be a target protein. In some cases, binding between an enzyme and its macromolecular ligand can modulate the activity of an enzyme with respect to its substrates.

A “macromolecular ligand” of a target protein is a naturally occurring molecule which binds to the surface of a target protein to form a complex which induces one or more biochemical events, as discussed in the previous paragraph. A macromolecular ligand can be a second protein, a carbohydrate, a nucleic acid, a glycoprotein, a lipoprotein, a nucleoprotein or a glycolipid.

Examples of target protein/macromolecular ligand interactions include protein ligand-receptor interaction; protein ligand-binding protein interaction; adhesion molecule interaction; protein-antibody interaction; complement component interaction; signal transduction protein interaction; protein transport; protein assembly; transcription and translation; cell secretion; and the like.

Suitable targeting groups bind non-covalently with the surface of a target protein with a Kd less than or equal to 1 mM, and preferably less than or equal to 100 μM. The targeting group binds at or in sufficient proximity to the binding site of the target protein and its macromolecular ligand such that formation of the complex between the target protein and its macromolecular ligand is inhibited by the compound. Targeting groups can be based on known inhibitors of protein/macromolecular ligand binding, provided that the inhibitor has the requisite binding affinity. Many such inhibitors are known in the art and include non-oligomeric (i.e., monomeric) molecules and oligomeric molecules such as polynucleotides, polypeptides and oligosaccharides. The targeting group can be based on known inhibitors that have been discarded as a drug candidates because their affinity for the target protein is too weak. Preferably, the targeting group is not liberated directly upon covalent binding of the reactive functional group to the target protein surface (i.e., is not a leaving group), although it may be degraded in vivo after such covalent binding, as discussed below. An example of one such inhibitor is shown below in Structural Formula (I). This inhibitor binds to the alpha subunit of the interleukin-2 receptor (target protein) with an Kd of about 3 μM and inhibits the binding of interleukin 2 (protein ligand of the receptor). The interleukin-2 receptor is found on T cells. This compound is described in Tilley et al., J. Am. Chem. Soc 119:7589 (1997), the entire teachings of which are incorporated herein by reference. Compounds which block the binding of interleukin-2 with its T cell receptor can be used as immunosuppressive agents to prevent rejection after organ transplant and to treat automimmune diseases such as rheumatoid arthritis, asthma, psoriasis and the like:

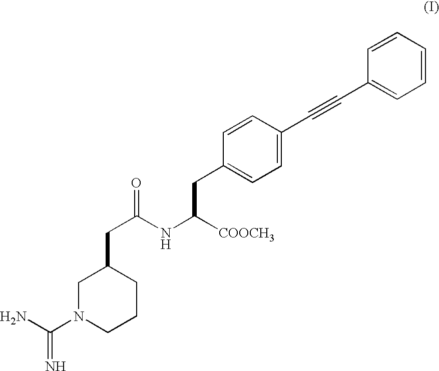

Alternatively, suitable targeting groups can be based on inhibitors identified through screening assays, e.g., high through-put screening of individual compounds or combinatorial libraries. Suitable assays detect inhibition of binding between the target protein and its macromolecular ligand and can be, for example, a binding assay (e.g., ELISA, radioreceptor binding assay, scintillation proximity assay, cell surface receptor binding assay, fluorescence energy transfer assay, surface plasmon resonance and HPLC), a biophysical assay or a functional assay. In yet another alternative, the targeting group is based on a molecule designed from a model of the target protein/macromolecular ligand complex. Methods of preparing such models are well known in the art and include computational models, x-ray crystal structures, structures obtained from nuclear magnetic resonance data and methods of binding site localization such as site directed mutagenesis. Based on the model, the binding site for the ligand is identified. In addition, it is possible to identify amino acids on the protein surface at or near the binding site which would have a non-covalent affinity for a suitable targeting group. From this data, a targeting group is designed which has functional groups suitably orientated in three dimensional space so as to positively interact with those surface amino acids. Optimization can be accomplished according to standard medicinal chemistry procedures, although it is contemplated that most targeting groups according to the present invention will be based on inhibitors with modest affinity for the target protein. A targeting group need not bind directly at the site at which the target protein and its ligand interact, but rather in sufficient proximity so that the complex formation between the target protein and its ligand is inhibited by the drug residue after covalent bond formation with the protein surface.

The function of the targeting group is to bind selectively to the proper target protein. However, the strong binding affinity (typically a Kd less than 10 nanomolar) necessary for a therapeutically effective inhibition of complex formation between the target protein and its ligand is largely a result of covalent bonding between the reactive functional group and the target protein surface. Therefore, targeting groups can be degradable in vivo, provided that the in vivo half life is sufficiently long so that the compound can reach its target. “Degradable” refers to chemically or enzymatically labile in vivo. The ability to use degradable target groups imparts the compounds of the present invention with a number of important advantages. Targeting groups can be based on molecules which would otherwise degrade too quickly to be useful drugs or might cause adverse biological reactions such as immune and allergic responses. Examples include carbohydrates, certain natural products, proteins, polypeptides, antibodies and monoclonal antibodies. Thus, in addition to small organic molecules (i.e., molecules with a molecular weight below about 1500 amu), the targeting group can be based on compounds having a molecular weight greater than 1500 amu, typically greater than 2000 amu, often greater than 3000 amu and preferably greater than 5000 amu.

An acceptable in vivo half-life is determined by the location of the target protein and the mode of administration. Short half-lives are acceptable if the target protein is quickly accessible by the selected route of administration. Longer half-lives are required as the time needed to reach the target increases. For example, an in vivo half-life of seconds is adequate, and in some cases preferable, if the target protein is located in the lungs and the drug is administered by inhalation. A slightly longer half-life in the range of minutes is adequate if the drug is administered systemically, for example, injected directly into the blood stream and the target protein is accessible from the circulatory or lymphatic system. Half-lives in the range of minutes to hours are required when the drug is administered orally and the target is an internal organ outside of the digestive system. The in vivo half-lives of the compounds of the present invention will generally be greater than one minute, typically greater than one hour and often greater than twelve hours. In vivo half-lives can be determined by standard pharmokinetic techniques, including evaluating the levels of the compound over time in blood or tissue samples by, e.g., HPLC, mass spectrometry or radiochemical techniques.

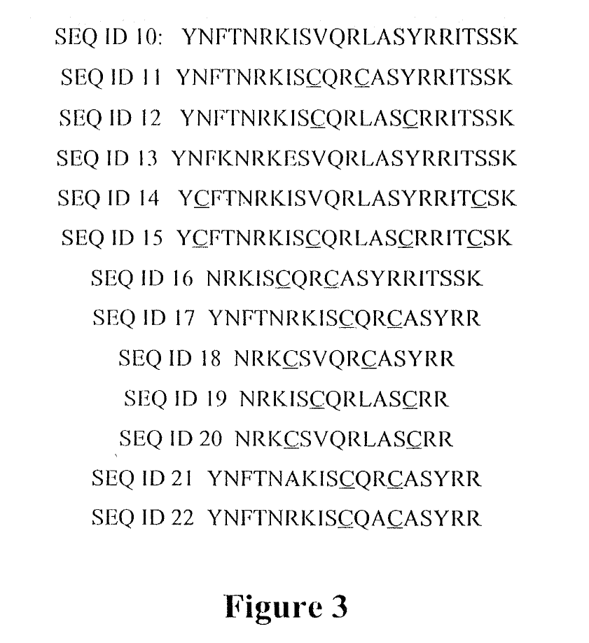

An example of a suitable degradable targeting group is based on polypeptides comprising portions of human monocyte chemoattractant protein-1 (MCP-1) (SEQ ID NO. 1: EICADPKQKWVQ; and SEQ ID NO. 2: EICLDPKQKWIQ). Another example of a suitable degradable targeting group is based on polypeptides comprising the ‘First Loop’ of MCP-1 (SEQ ID NO 3: AYNFTNRKISVQRLASYRRITSSK) or polypeptides comprising disulfide-cyclized derivatives of this region of MCP-1 (SEQ ID NO. 4: ACYNFTNRKISVQRLASYRRITSSKC; and SEQ ID NO. 5: YCFTNRKISCQRCASYRRITCSK) (intramolecular disulfide bonds occur between cysteinyl residues at positions 2 and 21 and positions 10 and 13). These peptides inhibit the binding between MCP-1 and the MCP-1 receptor (target protein). Derivatives of these peptides or other peptide fragments which bind to the MCP-1 receptor with sufficient affinity may also be used as targeting groups. Other suitable fragments of MCP-1 and derivatives of such fragments are provided by polypeptides having the amino acid sequence of SEQ ID NOs 10-22, shown in FIG. 3 and disclosed in Hemmerich et al., Biochemistry 38:13013 (1999). The peptides having the amino acid sequences of SEQ ID NO. 1 and SEQ ID NO. 2 are disclosed in Reckless and Grainger, Biochem J. 340:803 (1999). The peptides having the amino sequences of SEQ ID NO. 3 and SEQ ID NO. 4 are disclosed in Steitz et al., FEBS Letters 430:158 (1998). The peptides having the amino acid sequence of SEQ ID NO. 5 and SEQ ID NOS 10-22 are disclosed in Hemmerich et al., Biochemistry 38:13013 (1999). The entire teachings of these references are incorporated herein by reference. MCP-1 is essential for much of the pathology associated with autoimmune diseases such as asthma or rheumatoid arthritis. MCP-1 has also been identified as an important factor in the formation of atherosclerotic plaque. Inhibitors of the binding of MCP-1 and its receptor would be useful drugs in the treatment of these diseases.

As with fragments of MCP-1, suitable degradable targeting groups could be based on receptor binding fragments of other chemokines including, for example, MCP-2, MCP-3, MCP-4, MIP-1α, MIP-1β, RANTES, Eotaxin, MIP-3α, MIP-3β, MIP-4, MIP-5, SDF-1, fractalkine, IP-10, MIG, fMLP, NAP-2, I-309, TARC, HCC-1, GRO, ENA-78, GCP-2, platelet factor 4, lymphotactin, MDC, and IL-8; cytokines, growth factors, cell migration factors and interleukins, including, for example, IL-1α, IL-1β, IL-1ra, IL-2, IL-3, IL-4, IL-5, IL-6, IL-7, IL-9, IL-10, IL-11, IL-12, IL-13, IL-15, IL-16, IL-17, IL-18, FGF, TGF-α, TGF-β, PDGF, IGF, VEGF, EGF, keratinocyte growth factor, ECGF, heregulin, PLGF, endothelin, G-CSF, GM-CSF, erythropoietin, stem cell factor, IFNα, IFNβ, IFNγ, TNFα, TNFβ, NGF, apoptosis factors, CNTF, neurotrophins, bone morphogenic factors, ephrins and oncostatin; immunological receptors, for example, CD2, CD58, CD4, CD8, MHC class II, MHC class I, T cell antigen receptor, CD3, CD28, CTLA-4, B7-1, B7-2, CD40, CD40 Ligand (CD154), CD44, osteopontin, CD45, CD19, CD21, CD22 and Fc receptors; hormones, for example growth hormone, insulin, amylin, FSH, LH, MSH, TSH, prolactin, placental lactogen; adhesion molecules, for example, integrins, selectins and the Ig superfamily of adhesion molecules; complement components; immunoglobulins (e.g., IgA, IgE, IgG, IgM and the like); and the like. Compounds based on peptides, protein constructs, antibodies, carbohydrates, natural products, or small synthetic molecules that bind to receptors with sufficient affinity to serve as targeting groups for a receptor should also be included.

Alternatively, the chemokine can serve as the target protein by basing the targeting group on chemokine-binding fragments of the corresponding chemokine receptor. Examples include a peptide corresponding to the 35 amino-terminal residues of the MCP-1 receptor CCR2 (SEQ ID NO. 6: LSTSRSRFIRNTNESGEEVTTFFDYDYGAPCHKFD), a smaller peptide fragment comprising amino acids 19-32 from the same region of CCR2 (SEQ ID NO. 7: EVTTFFDYDYGAPC), or amino acids 9-23 from a homologous region from a viral chemokine that binds MCP-1, US28. (SEQ ID NO. 8: ELTTEFDYDDEATPC). One or both tyrosines in CCR2(19-32) or USB(9-23) may be chemically modified by phosphorylation or sulfation. Another example of a targeting group based on a chemokine binding fragment of a chemokine receptor is the peptide encompassing amino acids Pro21-Pro29 of the Interleukin-8 receptor referred to as CXCR1 (SEQ ID NO. 9: PPADEDYSP). Derivatives of these peptides or other peptide fragments which bind to MCP-1 or IL-8 with sufficient affinity may also be used as targeting groups. The peptide having the amino acid sequence of SEQ ID NO. 6 is disclosed in Monteclaro and Charo, J. Biol. Chem. 272:23186 (1997) and Charo et al., Proc. Natl. Acad. Sci. USA 91:2752 (1994). The peptides having the amino acid sequences of SEQ ID NO. 7 and SEQ ID NO. 8 are disclosed in Hemmerich et al., Biochemistry 38:13013 (1999). The peptide having the amino acid sequence of SEQ ID NO. 9 is disclosed in Skelton et al., Structure Fold. Des., 7:157 (1999). The entire teachings of these references are incorporated herein by reference.

Suitable targeting groups could be based upon chemokine binding fragments of other chemokine receptors, such as CCR1, CCR3, CCR4, CCR5, CCR6, CCR7, CCR8, CCR9, CCR10, CCR11, CXCR2, CXCR3, CXCR4, CXCR5 or binding fragments of other receptors such as those for interleukins, cytokines, growth factors, immune cell receptors, adhesion molecules, hormones, bone morphogenic proteins, complement components, immunoglobins, viral chemokine-binding proteins and the like. Compounds based on peptides, protein constructs, antibodies, natural products or small synthetic molecules that bind to the ligands with sufficient affinity to serve as targeting groups are also included.

Once a suitable inhibitor has been identified, it is modified to include an attaching group. The residue of the inhibitor following modification with the attaching group (and linker) is the targeting group. As noted earlier, the attaching group comprises an reactive functional group which is preferably weakly reactive. Examples include alcohols, alkyl halides, sulfonates, sulfonamides, phosponates, boronic acids, boronic esters, alkoxysilanes, aryloxysilanes, acyloxysilanes, oximes, hydroxyamides, hydroxyimides, ethers, cyclic ethers, epoxides, amines, aziridines, quaternary ammonium salts, thiols, thioethers, cyclic thioethers, episulfides, sulfonium salts, disulfides, N-alkylthio-amides, N-arylthio-amides, ylids, phosphorous ylids, carboxylic acids, esters, thioesters, lactones, beta-lactones, orthoesters, amides, thioamides, lactams, beta-lactams, dialkoxyamide acetals, imides, azalactones, imidates, amidates, aldehydes, acetals, thioacetals, ketones, ketals, thioketals, imines, iminium salts, 1,2-dicarbonyls (aldehyde, ketone, carboxylic acids and derivatives), 1,3-dicarbonyls (aldehyde, ketone, carboxylic acids and carboxylic acid derivatives such as amides and esters), alpha,beta-unsaturated carbonyls (aldehyde, ketone, carboxylic acids and carboxylic acid derivatives such as amides and esters), non-aromatic heterocyclic groups (including non-aromatic heterocyclic groups with alkylated or acylated heteroatoms in the ring), aromatic heterocyclic groups (including aromatic heterocyclic groups with alkylated or acylated heteroatoms in the ring), organometallic groups (suitable metals include platinum, palladium, nickel, copper, iron, zinc, manganese, aluminium, magnesium or calcium) and the like.

In most cases, the targeting group is connected to the attaching group through a linking group. The attaching group or linking group is attached to a position in the targeting group which does not significantly adversely affect the ability of the targeting group to bind with the target protein. Suitable positions can be identified by modeling the complex formed from the inhibitor and the targeting protein by computational means, x-ray crystallography NMR data, or site directed mutageneis, as described above. Alternatively, suitable positions can be identified empirically, for example, by preparing analogs of the inhibitor by systematically modifying various positions and then assaying the ability of each to inhibit binding between the target protein and its ligand.

As noted earlier, covalent bond formation between the reactive functional group and a compatible functional group on the target protein surface is dependent on the reactive functional group occupying a suitable position in three dimensional space subsequent to targeting group/target protein binding. A suitable reactive functional group and its proper position can be determined from a model of the target protein/ targeting group complex, generated as described above. The reactive functional group can be brought into the proper position by an appropriate selection of the length and type of linker group and the position at which the linker is attached to the targeting group.

Alternatively, a suitable linking group (length and orientation) and reactive functional group can be selected empirically, for example, by preparing analogs of the inhibitor and systematically varying the position and length of the linking group and/or the type of reactive functional group. The ability of each analog to affect binding between the target protein and its ligand and to covalently bind with the target protein is assayed. Analogs which covalently bind to the target protein and which show increased inhibition of binding between the target protein and its ligand are selected for further testing and or further optimization, for example, by preparing another series of analogs. The optimization cycle can be repeated as many times as needed.

In one aspect, the linking group is inert, i.e., substantially unreactive in vivo, e.g., is not chemically or enzymatically degraded. Examples of inert groups which can serve as linking groups include aliphatic chains such as alkyl, alkenyl and alkynyl groups (e.g., C1-C20), cycloalkyl rings (e.g., C3-C1O), aryl groups (carbocyclic aryl groups such as 1-naphthyl, 2-naphthyl, 1-anthracyl and 2-anthracyl and heteroaryl group such as N-imidazolyl, 2-imidazole, 2-thienyl, 3-thienyl, 2-furanyl, 3-furanyl, 2-pyridyl, 3-pyridyl, 4-pyridyl, 2-pyrimidy, 4-pyrimidyl, 2-pyranyl, 3-pyranyl, 3-pyrazolyl, 4-pyrazolyl, 5-pyrazolyl, 2-pyrazinyl, 2-thiazole, 4-thiazole, 5-thiazole, 2-oxazolyl, 4-oxazolyl, 5-oxazolyl, 2-benzothienyl, 3-benzothienyl, 2-benzofuranyl, 3-benzofuranyl, 2-indolyl, 3-indolyl, 2-quinolinyl, 3-quinolinyl, 2-benzothiazole, 2-benzooxazole, 2-benzimidazole, 2-quinolinyl, 3-quinolinyl, 1-isoquinolinyl, 3-quinolinyl, l-isoindolyl, and 3-isoindolyl), non-aromatic heterocyclic groups (e.g., 2-tetrahydrofuranyl, 3-tetrahydrofuranyl, 2-tetrahyrothiophenyl, 3-tetrahyrothiophenyl, 2-morpholino, 3-morpholino, 4-morpholino, 2-thiomorpholino, 3-thiomorpholino, 4-thiomorpholino, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl and 4-thiazolidinyl) and aliphatic groups in which one, two or three methylenes have been replaced with —O—, —S—, —NH—, —SO₂—, —SO— or —SO₂NH—. Optionally, the linking group (or attaching group) can be further substituted with one or more additional groups referred to as “blocking groups”. Blocking groups should not adversely affect the ability of the target group to non-covalently bind and with the reactive functional group to covalently bind with the target protein surface. The blocking group increases the ability of the drug to inhibit target protein/macromolecular ligand binding after covalent modification of the target protein with the drug. The blocking group can add steric bulk to the drug and thereby increase the drug's inhibitory ability by physically blocking access to the target protein/macromolecular ligand binding site. In this instance, the blocking group can be, for example, an aliphatic group, aryl group or non-aromatic heterocyclic group. Alternatively, the blocking group can be a charged or polar group which can electronically repel a macromolecular ligand from the binding site. Examples include carboxylic acids, amines and hydroxyl groups.

Examples of inert linking groups are shown below in Structures (II)-(IV):

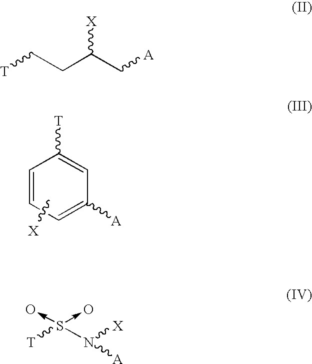

In Structural Formulas (II)-(IV), T is targeting group; A is an attaching group; and X is —H or a blocking group.

In another aspect, the linking group is enzymatically or chemically labile in vivo. Examples include groups which comprise ester and amide bonds, which can be cleaved under enzymatic or mildly basic conditions; ester, oxime, and acetal groups (including carbohydrate derivatives) which can be cleaved under mildly acidic conditions; and hydroquinone, quinone and disulfide analogs, which can be cleaved under redox or radical conditions. Examples are shown below in Structural Formulas (V)-(VIII):

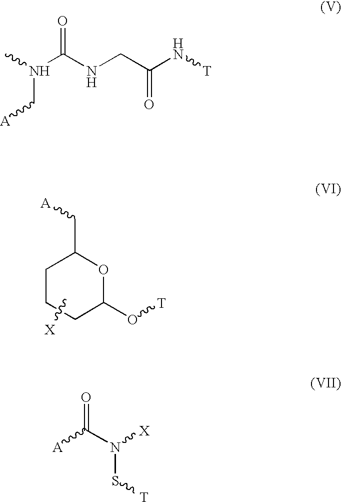

X, A and T are as described above for Structural Formulas (II)-(IV). The use of cleavable linking groups allows the use of targeting groups having a wide range of size, from protein constructs to small synthetic molecules. The use of a cleavable linking group has the same advantages as degradable targeting groups, discussed herein above. To enhance inhibition, the linker (or attaching group) can advantageously include a blocking group in a part of the linker which remains bound to the target protein after cleavage.

A “subject” is preferably a mammal, such as a human, but can also be an animal in need of veterinary treatment, e.g., domestic animals (e.g., dogs, cats and the like), farm animals (e.g., cows, sheep, pigs, horses and the like) and laboratory animals (e.g., rats, mice, guinea pigs and the like).

An “effective amount” of a compound is a quantity sufficient to achieve a desired therapeutic and/or prophylactic effect, such as an amount which results in the prevention of or a decrease in the symptoms associated with a disease that is being treated. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compound can range from about 0.1 mg per day to about 100 mg per day for an adult. Preferably, the dosage ranges from about 1 mg per day to about 100 mg per day. The compounds of the present invention can also be administered in combination with one or more additional therapeutic agents.

The compound can be administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compound can also be administered orally (e.g., dietary), topically, by inhalation (e.g., intrabronchial, intranasal, oral inhalation or intranasal drops), by oral mucosa or rectally, depending on the disease or condition to be treated.

The compound can be administered to the individual in conjunction with an acceptable pharmaceutical carrier as part of a pharmaceutical composition. Formulation of a compound to be administered will vary according to the route of administration selected (e.g., solution, emulsion, capsule). Suitable pharmaceutical carriers may contain inert ingredients which do not interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

Binding between proteins and their macromolecular ligands and the inhibition thereof can be assayed by any suitable method, including binding assays, biophysical assays and functional assays. Dissociation constants, “Kds,” can be assessed by binding assays or biophysical assays using methods known in the art. Kd values recited in the present application refer to values obtained at physiological ionic strength and physiological pH and with reagents (including target proteins) that are substantially free of impurities that would affect the numerical value determined by the assay. For soluble target proteins, the Kd values recited herein are obtained by biophysical assays. For insoluble target proteins, the Kd values recited herein are obtained from binding assays with immobilized target proteins in, e.g., subcellular preparations or detergent extracts.

A binding assay refers to mixing the protein, its macromolecular ligand and a test compound under conditions suitable for binding between the protein and the ligand and assessing the amount of binding between the protein and its ligand. The amount of binding is compared with a suitable control, which can be the amount of binding in the absence of the test compound, the amount of the binding in the presence of a known inhibitor, or both. The amount of binding can be assessed by any suitable method. Binding assay methods include, for example, ELISA, radioreceptor binding assays, scintillation proximity assays, cell surface receptor binding assays, fluorescence energy transfer assays, liquid chromatography, membrane filtration assays, and the like. Biophysical assays for the direct measurement of compound binding to the target protein include, for example, nuclear magnetic resonance, fluorescence, fluorescence polarization, surface plasmon resonance (BIACOR chips) and the like. Conditions suitable for binding between the protein and its ligand will depend on the protein and its ligand and can be readily determined by one of ordinary skill in the art.

Biophysical assays assess the binding of a compound to a protein target by measuring the change in some biophysical property of the compound or target protein before and after binding. The degree of change in the biophysical property correlates to the degree of binding. Examples of tools which measure biophysical properties include, NMR spectroscopy, ultraviolet/visible spectroscopy, fluorescence and surface plasmon resonance (BIACOR chips).

A functional assay refers to an assay which assesses binding between a protein and its macromolecular ligand by measuring the degree of a biological response which results from the binding. For example, binding between certain receptor proteins and their ligands will cause the modulation of an intracellular messenger such as cyclic AMP, cell secretion, cell division, cell migration, cell death, nucleic acid synthesis, protein synthesis, chemical modification of proteins such as phosphorylation or dephosphorylation, calcium flux and the like. To carry out a functional assay generally requires a cell which expresses the protein or its ligand or provides a mixture that includes the protein, its ligand and the biological molecules involved in the biological response. To carry out the assay, the protein, its macromolecular ligand and a test compound are mixed under conditions suitable for binding for effecting the biological response. The degree of the biological response is assessed and compared with a suitable control, which is the same mixture without the test compound.

The kinetic rate for a given reaction is determined by following the disappearance of reactant(s) and/or the formation of reaction product(s). The most general and widely applied methods employ spectroscopic techniques which can continuously monitor the extent of the reaction by observing changes in concentration. However, any property that can be measured and related to the concentration of a reactant or product would suffice to determine a reaction rate (e.g., pH measurements, conductance measurements, optical rotation). The determination of the reaction rates can be assessed by any suitable method known to one of ordinary skill in the art. These techniques includes, for example, high-pressure liquid chromatography (HPLC), fourier transform infrared spectroscopy (FT-IR), ultraviolet/visible spectroscopy (UV/VIS), fourier transform nuclear magnetic resonance (FT-NMR) and the like. Forward reaction rates are determined at physiological conditions, which include physiological pH and physiological ionic strength.

Covalent binding between a protein target and an inhibitor can be determined by a suitable method known to one of ordinary skill in the art. One suitable method is described in Weir et al., Biochemistry 37:6645 (1998) and includes isolating the product of the reaction between the inhibitor and target protein and analyzing the product by electrospray ionization mass spectrometry. The molecular ion peaks will indicate whether binding is covalent. The entire teachings of Wier et al. is incorporated herein by reference. Covalent binding can be assessed by other techniques known in the art, including x-ray crystal structure of the target protein/drug complex; by NMR, for example, by analysis of the change in chemical shifts in the drug after binding with the target protein; and by capillary electrophoresis, for example, by analysis of the change in mobility of the protein following covalent attachment of the drug.

Another embodiment of the present invention is a method of detecting a target protein in a sample or assessing the quantity of a target protein in a sample. The method comprises the step of combining the sample with a compound comprising a targeting group which binds non-covalently to a surface of the target protein, an attaching group comprising a reactive functional group which covalently binds to an amino acid on the surface of the protein after non-covalent binding between the targeting group and the target protein, and, optionally, a linker group. Preferably, the compound can inhibit binding between the target protein and one of its macromolecular ligands. The combination is made under conditions suitable for non-covalent binding between the targeting group and the target protein and for covalent binding between the reactive functional group and protein surface, thereby forming a covalent complex between the target protein and the compound. The quantity of the complex is then assessed and compared with a suitable control, e.g., the amount of complex formed in a similar sample known to be devoid of the the target protein. The amount of complex formed in the control can be determined simultaneously with or subsequent to assessment of the sample, or, alternatively, can be predetermined. The level of complex formed can be determined by standard methods, e.g., using radiolabeled, flourescently or spin labeled compound, HPLC or capillary electrophoresis. A greater level of complex formation in the sample compared with the control is indicative of the presence of the target protein in the sample.

The method of detecting a target protein in a sample can be used as a method of diagnosis for a subject suspected of having a disease characterized by an overabundance (or underabundance) of a target protein in a tissue or blood sample. The level of the target protein in a blood or tissue sample obtained from a subject is determined and compared with the level found in a blood sample or a sample from the same tissue type obtained from an individual who is free of the disease. An overabundance (or underabundence) of the target protein in the sample obtained from the subject suspected of having the disease compared with the sample obtained from the healthy subject is indicative of the disease in the subject being tested. Further testing may be required to make a positive diagnosis.

There are a number of diseases in which the degree of overexpression (or underexpression) of certain target proteins, referred to herein as “prognostic proteins”, is known to be indicative of whether a subject with the disease is likely to respond to a particular type of therapy or treatment. Thus, the method of detecting a target protein in a sample can be used as a method of prognosis, e.g., to evaluate the likelihood that the subject will respond to the therapy or treatment. The level of the relevant prognostic protein in a suitable tissue or blood sample from the subject is determined and compared with a suitable control, e.g., the level in subjects with the same disease but who have responded favorably to the treatment. The degree to which the prognostic protein is overexpressed (or underexpressed) in the sample compared with the control may be predictive of likelihood that the subject will not respond favorably to the treatment or therapy. The greater the overexpression (or underexpression) relative to the control, the less likely the subject will respond to the treatment.

There are a number of diseases in which the degree of overexpression (or underexpression) of certain target proteins, referred to herein as “predictive proteins”, is known to be indicative of whether a subject will develop a disease. Thus, the method of detecting a target protein in a sample can be used as a method of predicting whether a subject will develop a disease. The level of the relevant predictive protein in a suitable tissue or blood sample from a subject at risk of developing the disease is determined and compared with a suitable control, e.g., the level in subjects who are not at risk of developing the disease. The degree to which the predictive protein is overexpressed (or underexpressed) in the sample compared with the control may be predictive of likelihood that the subject will develop the disease. The greater the overexpression (or underexpression) relative to the control, the more likely the subject will development the disease.

The levels of certain proteins in a particular tissue (or in the blood) of a subject may be indicative of the toxicity, efficacy, rate of clearance or rate of metabolism of a given drug when administered to the subject. The methods described herein can also be used to determine the levels of such protein(s) in subjects to aid in predicting the response of such subjects to these drugs.

The methods of the present invention can also be used to assess whether an individual expresses a target protein or a polymorphic form of the target protein in instances where a compound of the present invention has greater affinity for the target protein for its polymorphic form (or vice versa).

The sample can be a biological sample such as a tissue or blood sample from an individual.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of identifying a compound which covalently binds to the surface of a target protein in sufficient proximity to the binding site between a macromolecular ligand and the target protein to inhibit binding of the macromolecular ligand with the target protein, said method comprising the steps of:
a) selecting a lead compound which non-covalently binds to the surface of a target protein with a Kd of greater than about 0.10 μM, wherein said lead compound is represented by the structural formula T-H;
b) preparing a plurality of analogs of the lead compound, each analog being represented by the structural formula T-L-A, wherein L is an inert linking group, A is a moiety comprising a reactive functional group and -L-A, taken together, is different for each analog;
c) combining the target protein, macromolecular ligand and each analog under conditions suitable for binding between the target protein and macromolecular ligand;
d) assaying each combination of step c) for inhibition of macromolecular ligand/target protein binding and for covalent binding between the analog and the target protein; and
e) selecting analogs which inhibit macromolecular ligand/target protein binding and which covalently bind with the target protein.

2. The method of claim 1 wherein the lead compound inhibits binding of the macromolecular ligand with the target protein.

3. The method of claim 2 additionally comprising the steps of:
f) preparing a plurality of additional analogs of an analog selected in step e);
g) combining the target protein, macromolecular ligand and each additional analog under conditions suitable for binding between the target protein and macromolecular ligand;
h) assaying each combination of step g) for inhibition of macromolecular ligand/target protein binding and for covalent binding between the additional analog and the target protein; and
i) selecting additional analogs with improved inhibition of macromolecular ligand/target protein binding compared with the analog selected in step e).

4. The method of claim 3 additionally comprising the step of repeating steps f)-h) with an analog selected in step i) and selecting analogs with improved inhibition of macromolecular ligand/target protein binding compared with the analog selected in step i).

5. The method of claim 2 wherein the lead compound is selected by screening a combinatorial library of compounds for inhibition of target protein/macromolecular ligand interaction.

6. The method of claim 2 wherein the complex between the target protein and macromolecular ligand is modeled computationally, by x-ray crystallography; by nuclear magnetic resonance spectrophotometry or by active site localization; the target protein/macromolecular ligand binding site is identified from the model(s); and wherein a lead compound is designed based on its ability to bind to the protein target/macromolecular ligand binding site.

7. The method of claim 2 additionally comprising the steps of:
a) modeling the complex between the target protein and the lead compound computationally, by x-ray crystallography; by nuclear magnetic resonance spectrophotometry or by active site localization;
b) identifying reactive functional groups on the surface of the target protein in the vicinity of the binding site between the target protein and lead compound; and
c) selecting A groups that can form covalent bonds with the reactive functional groups on the surface of the protein; and
d) selecting L groups that will bring the A groups into sufficient proximity with the reactive functional groups on the surface of the protein to covalently react after binding between the targeting group and the target protein.

8. The method of claim 2 wherein the reactive group has a reactivity with the corresponding free amino acid under physiological conditions of less than about 10⁻⁴ M⁻¹sec⁻¹.

9. The method of claim 2 wherein the linking group is inert.

10. The method of claim 2 wherein said linking group is cleavable in vivo.

11. The method of claim 2 wherein the targeting group is degradable in vivo.

12. The method of claim 10 or 11 wherein the compound has an in vivo half-life of at least about one minute.

13. The method of claim 8 wherein the targeting group is a carbohydrate, natural product, peptide, protein, antibody or monoclonal antibody.

14. A method of identifying a compound which covalently binds to the surface of a target protein in sufficient proximity to the binding site between a macromolecular ligand and the target protein to inhibit binding of the macromolecular ligand with the target protein, said method comprising the steps of:
a) selecting a lead compound which non-covalently binds to the surface of a target protein, wherein said lead compound is represented by the structural formula T-H;
b) preparing a plurality of analogs of the lead compound, each analog being represented by the structural formula T-L-A, wherein L is a linking group, A is a moiety comprising a reactive functional group, -L-A, taken together, is different for each analog and the linking group is cleavable in vivo or the targeting group is degradable in vivo;
c) combining the target protein, macromolecular ligand and each analog under conditions suitable for binding between the target protein and macromolecular ligand;
d) assaying each combination of step c) for inhibition of macromolecular ligand/target protein binding and for covalent binding between the analog and the target protein; and
e) selecting analogs which inhibit macromolecular ligand/target protein binding and which covalently bind with the target protein.

15. The method of claim 14, wherein the lead compound inhibits binding of the macromolecular ligand with the target protein.

16. The method of claim 15, additionally comprising the steps of:
f) preparing a plurality of additional analogs of an analog selected in step e);
g) combining the target protein, macromolecular ligand and each additional analog under conditions suitable for binding between the target protein and macromolecular ligand;
h) assaying each combination of step g) for inhibition of macromolecular ligand/target protein binding and for covalent binding between the additional analog and the target protein; and
i) selecting additional analogs with improved inhibition of macromolecular ligand/target protein binding compared with the analog selected in step e).

17. The method of claim 16 additionally comprising the step of repeating steps f)-h) with an analog selected in step i) and selecting analogs with improved inhibition of macromolecular ligand/target protein binding compared with the analog selected in step i).

20. The method of claim 15 wherein the lead compound is selected by screening a combinatorial library of compounds for inhibition of target protein/macromolecular ligand interaction.

19. The method of claim 15 wherein the complex between the target protein and macromolecular ligand is modeled computationally or by x-ray crystallography; the target protein/macromolecular ligand site is identified from the model(s); and wherein a lead compound is designed based on its ability to bind to the protein target/macromolecular ligand binding site.

20. The method of claim 15, additionally comprising the steps of:
a) modeling the complex between the target protein and lead compound computationally, by x-ray crystallography; by nuclear magnetic resonance spectrophotometry or by active site localization;
b) identifying reactive functional groups on the surface of the target protein in the vicinity of the binding site between the target protein and lead compound; and
c) selecting groups that can form covalent bonds with the reactive functional groups on the surface of the protein; and
d) selecting L groups that will bring the A groups into sufficient proximity with the reactive functional groups on the surface of the protein to covalently react after binding between the targeting group and target protein.

21. The method of claim 15 wherein the compound has an in vivo half-life of at least about one minute.

22. The method of claim 15 wherein the linking group is inert.

23. The method of claim 21 wherein the compound has a molecular weight greater than about 1500 amu.

24. The method of claim 21 wherein the targeting group binds non-covalently to a surface of the target protein with a Kd of greater than about 0.10 μM.

25. The method of claim 24 wherein the reactive group has a reactivity with the corresponding free amino acid under physiological conditions of less than about 10⁻⁴ M⁻¹sec⁻¹.

26. The method of claim 21 wherein the targeting group is a carbohydrate, natural product, peptide, protein, antibody or monoclonal antibody.

27. A compound for inhibiting binding between a target protein and a macromolecular ligand of the target protein, said compound comprising a targeting group and an attaching group, wherein:
the targeting group is a moiety that binds non-covalently to a surface of the target protein with a Kd of greater than about 0.10 μM and within sufficient proximity to the target protein/macromolecular ligand binding site such that the compound inhibits binding between the target protein and the macromolecular ligand; and
the attaching group is a moiety comprising a reactive functional group which can form a covalent bond with an amino acid on the surface of the target protein after the targeting group binds with the target protein.

28. The compound of claim 27 wherein the reactive functional group has a reactivity with the corresponding free amino acid under physiological conditions of less than about 10⁻⁴ M⁻¹sec⁻¹.

29. The compound of claim 27 additionally comprising an inert linking group which connects the targeting group with the attaching group.

30. The compound of claim 27 additionally comprising a linking group which connects the targeting group with the attaching group, wherein said linking group is cleavable in vivo.

31. The compound of claim 27 wherein the targeting group is degraded in vivo.

32. The compound of claim 30 or 31 wherein the compound has an in vivo half-life of at least about one minute.

33. The compound of claim 27 wherein the compound has a molecular weight greater than about 1500 amu.

34. The compound of claim 33 wherein the targeting group is a carbohydrate, natural product, peptide, protein, antibody or monoclonal antibody.

35. A compound for inhibiting binding between a target protein and a macromolecular ligand of the target protein, said compound comprising a targeting group, an attaching group and, optionally, a linking group, wherein:
the targeting group is a moiety that binds non-covalently to a surface of the target protein and within sufficient proximity to the target protein/macromolecular ligand binding site such that the compound inhibits binding between the target protein and the macromolecular ligand;
the attaching group is a moiety comprising a reactive functional group which can form a covalent bond with an amino acid on the surface of the target protein after the targeting group binds with the target protein;
the linking group connects the targeting group and the attaching group; and
the targeting group is degradable in vivo or the linking group is cleavable in vivo.

36. The compound of claim 35 wherein the compound has an in vivo half-life of at least about one minute.

37. The compound of claim 36 wherein the compound comprises an inert linking that connects the targeting group and attaching group.

38. The compound of claim 36 wherein the compound has a molecular weight greater than about 1500 amu.

39. The compound of claim 38 wherein the targeting group is a carbohydrate, natural product, peptide, protein, antibody or monoclonal antibody.

40. The compound of claim 36 wherein the targeting group binds non-covalently to a surface of the target protein with a Kd of greater than about 0.10 μM.

41. The compound of claim 40 wherein the reactive functional group has a reactivity with the corresponding free amino acid under physiological conditions of less than about 10⁻⁴ M⁻¹sec⁻¹.

42. A method of inhibiting binding between a target protein and a macromolecular ligand in a subject in need of such inhibition, said method comprising the step of administering to the subject an effective amount of a compound comprising a targeting group and an attaching group, wherein:
the targeting group is a moiety that binds non-covalently to a surface of the target protein with a Kd of greater than about 0.10 μM and within sufficient proximity to the target protein/macromolecular ligand binding site such that the compound inhibits binding between the target protein and the macromolecular ligand; and
the attaching group is a moiety comprising a reactive functional group which can form a covalent bond with an amino acid on the surface of the target protein after the target group binds with the target protein.

43. The method of claim 42 wherein the reactive functional group has a reactivity with the corresponding free amino acid under physiological conditions of less than about 10⁻⁴ M⁻¹sec⁻¹.

44. The method of claim 42 additionally comprising an inert linking group which connects the targeting group with the attaching group.

45. The method of claim 42 additionally comprising a linking group which connects the targeting group with the attaching group, wherein said linking group is cleavable in vivo.

46. The method of claim 42 wherein the targeting group is degraded in vivo.

47. The method of claim 45 or 46 wherein the compound has an in vivo half-life of at least about one minute.

48. The method of claim 42 wherein the compound has a molecular weight greater than about 1500 amu.

49. The method of claim 48 wherein the targeting group is a carbohydrate, natural product, peptide, protein, antibody or monoclonal antibody.

50. A method of inhibiting binding between a target protein and a macromolecular ligand in a subject in need of such inhibition, said method comprising the step of administering to the subject an effective amount of a compound comprising a targeting group and an attaching group, wherein:
the targeting group is a moiety that binds non-covalently to a surface of the target protein and within sufficient proximity to the target protein/macromolecular ligand binding site such that the compound inhibits binding between the target protein and the macromolecular ligand;
the attaching group is a moiety comprising a reactive functional group which can form a covalent bond with an amino acid on the surface of the target protein after the targeting group binds with the target protein;
the linking group connects the targeting group and the attaching group; and
the targeting group is degradable in vivo or the linking group is cleavable in vivo.

51. The method of claim 50 wherein the compound has an in vivo half-life of at least about one minute.

52. The method of claim 51 wherein the compound comprises an inert linking group that connects the targeting group and the attaching group.

53. The compound of claim 51 wherein the compound has a molecular weight greater than about 1500 amu.

54. The method of claim 53 wherein the targeting group is a carbohydrate, natural product, peptide, protein, antibody or monoclonal antibody.

55. The method of claim 51 wherein the targeting group binds non-covalently to a surface of the target protein with a Kd of greater than about 0.10 μM.

56. The method of claim 55 wherein the reactive functional group has a reactivity with the corresponding free amino acid under physiological conditions of less than about 10⁻⁴ M⁻¹sec⁻¹.
